(19) United States
(12) Patent Application Publication
(10) Pub. No.: US 2026/0325114 A1
(43) Pub. Date: Oct. 22, 2026

(54) Method and Apparatus for Integrated Breast Biopsy and Clip Placement

(71) Applicant: SAMIR PATEL, GRANGER, IN (US)

(72) Inventor: SAMIR PATEL, GRANGER, IN (US)

(21) Appl. No.: 19/650,584

(22) Filed: Apr. 17, 2026

Related U.S. Application Data
(60) Provisional application No. 63/791,750, filed on Apr. 21, 2025.
(60) Provisional application No. 63/797,418, filed on Apr. 30, 2025.

(51) Int. Cl. A61B 10/00; A61B 10/02; A61B 90/00

(52) U.S. Cl. CPC A61B 10/0041; A61B 10/0275; A61B 2010/0208; A61B 2090/3908; A61B 2090/3987

(57) ABSTRACT

A biopsy and marker procedure for a single needle penetration per tissue abnormality. A needle pierces the body led by a sharpened distal end and a biopsy trough. The needle has an at least partially hollow interior passage fluidly connected to the trough. The trough of the needle is positioned near a tissue abnormality under guidance of an imaging modality. A tissue sample is cut from the tissue abnormality and transferred to a specimen container fluidly connected to the interior passage. A marker clip is deployed through the trough without withdrawing the needle. The marking clip deploys at least partially through the hollow interior passage of the needle.

### CROSS-REFERENCE TO RELATED APPLICATIONS

This application claims priority to U.S. Provisional Patent Application Ser. No. 63/791,750 filed 21 Apr. 2025, titled “Method and Apparatus for Integrated Breast Biopsy and Clip Placement”, and also claims priority to U.S. Provisional Patent Application Ser. No. 63/797,418 filed 30 Apr. 2025, titled “Method and Apparatus for Integrated Breast Biopsy and Clip Placement”.

### BACKGROUND OF THE INVENTION

Once a common technique, fine needle aspiration (FNA) is now used far less frequently. FNA cannot determine if cancer is invasive or provide tumor markers, and it requires specialty training for interpretation. FNA also has a substantial rate of insufficient samples.

As summarized by one set of authors (O'Flynn E A, Wilson A R, Michell M J. Image-Guided Breast Biopsy: State-of-the-Art. Clinical Radiology. 2010; 65(4): 259-70), “There has been steady development in percutaneous biopsy techniques since the early 1990s, driven by the large numbers of women undergoing assessment for nonpalpable lesions detected by mammographic screening. Fine-needle aspiration cytology (FNA) was the original method of sampling and has been used for diagnosis since the 1950s. This was followed in the 1990s by increasing use of core needle biopsy (CB). The accuracy of both has been improved by ultrasound and stereotactic guidance. Larger bore vacuum-assisted biopsy (VAB) devices then became available in the late 1990s and are now commonplace” in most breast centers and health care facilities.

The development of advanced imaging techniques permits precise localization and delineation of the extent of breast lesions. This leads to improved patient management options, often resulting in more targeted surgical excisions. Thus, image-guided breast biopsy is an integral component to the current standard of care for breast abnormality assessment. The types of imaging guidance for breast biopsies include stereotactic, ultrasound (US), and magnetic resonance (MR). This has many advantages over open surgical biopsy (lower cost, lower patient morbidity, faster patient recovery, and minimal to no scarring).

As summarized by another set of authors (Huppe A I, Brem R F. Minimally Invasive Breast Procedures: Practical Tips and Tricks. AJR. American Journal of Roentgenology. 2020;214(2): 306-315), “Percutaneous, image-guided breast procedures are widely accepted as the standard of care by the American Society of Breast Surgeons and the American College of Radiology (ACR) for the pathologic diagnosis of both palpable and nonpalpable breast lesions, with studies showing equal efficacy to open surgical biopsies [1-8]. In addition, minimally invasive biopsies are less invasive and less costly, and they have less complications with faster recovery times and little to no scarring [5, 9-11].” Huppe et al. also note that “The use of directional, vacuum-assisted biopsy devices for ultrasound-guided procedures can be helpful for small or subtle lesions that may become difficult to visualize after initial sampling.”

Radiologists commonly perform most image-guided breast biopsies. However, image-guided breast biopsies are also performed by surgeons. Approximately 1 in 8 women (13%) in the United States will develop invasive breast cancer at some point in their life (from https://www.breastcancer.org/facts-statistics). 272,454 new breast cancers were diagnosed in 2021, and 279,731 new breast cancers were diagnosed in the United States in 2022 (from https://www.cdc.gov/united-states-cancer-statistics/publications/breast-cancer-stat-bite.html). Recent predictions for 2024 suggest a 14% increase in new breast cancer diagnoses from 2021, estimating that “In 2024, it is estimated that there will be 310,720 new cases of female breast cancer” (from https://seer.cancer.gov/statfacts/html/breast-subtypes.html). Most non-surgical image-guided breast biopsies (60-80%) do not result in a diagnosis of cancer. Some estimates are that between 900,000 to 1.8 million image-guided breast biopsies might have been performed in 2024.

Several devices are available, each with their own minor technical differences. They are adaptable to be used under, for example, ultrasound, stereotactic, or magnetic resonance imaging (MRI) guidance. The non-surgical image-guided breast biopsy procedures currently used to determine if an imaging abnormality represents cancer typically involves two separate devices. The first is a tissue acquisition biopsy device, and the second is a marker clip placement device. The standard of care for image-guided breast biopsies typically requires placing a marker clip after tissue removal for future location identification. Such identification might be for surgically removing breast tissue at such location. More than 85% of surgically treated breast cancers are diagnosed with image-guided core needle biopsy prior to surgery (Gilmore R C, Wang P, Kaczmarski K, Hutfless S, Euhus D M, Jacobs L K, Habibi M, Lange J, Camp M, Makary M A. Underutilization of Needle Biopsy Before Breast Surgery: A Measure of Low-Value Care. Ann Surg Oncol. 2021 May; 28(5):2485-2492).

Past and existing breast core needle biopsy involves the use of a biopsy device to remove tissue from the breast to diagnose malignant and benign breast pathologies. The biopsy device is placed with its tip in the breast tissue at or adjacent to an abnormality requiring tissue diagnosis. An abnormality is a finding or detection of a suspect area potentially situated for breast cancer. The average size of an abnormality varies and a study on ultrasound-guided vacuum-assisted biopsies found that the average size of excised lesions was 15.8 mm, with a range from 5 mm to 55 mm (see Breast J. 2011 September-October; 17(5): 490-7). An abnormality may be diagnosed following detection on a mammogram of a mass, calcifications, architectural distortion, asymmetric finding, or a combination of these findings. A tissue diagnosis via biopsy is often prescribed to confirm the presence (or absence) of an invasive cancer, in situ cancer, or a nonmalignant process, as the findings from a mammogram are often not pathognomonic. After completion of the biopsy (tissue removal), the biopsy device is partially or completely removed. Then a separate device is placed through the pathway created by the biopsy device. This separate device has a marker clip at its distal end. This device is positioned with its tip at the biopsied site within the breast. Then a marker clip is deployed into the breast through the separate device, and the marker clip remains in the breast (possibly permanently). Marker clip placement after breast biopsy is the standard of care, particularly for when a biopsied breast abnormality needs to be removed in a further procedure (e.g., because of cancer), or to identify a non-cancerous abnormality in the future when the patient undergoes breast imaging to avoid future breast biopsy of the same pre-existing abnormality.

In view of the millions of annual procedures, even incremental innovations potentially result in a cumulatively large improvement for the patient population. The importance to a patient of any incremental improvement is often evident in a patient's interaction. For example, when discussing the procedure with patients, some physicians formulate language to limit the patient's anxiety, such as the term “biopsy device” instead of “needle.” The preference is to make the patient an active participant, with explanations of what the patient should expect during the biopsy and when the biopsy results are expected to be available. Over the last few decades, while there have been some key changes to the approach to breast investigation and disease management, further innovation and improvements are needed. Thus, there remains a need for an improvement or improvements to imaging and interventional therapy that positively impact one or more aspects such as patient experience, physician satisfaction, procedural accuracy and safety.

### SUMMARY OF THE INVENTION

One embodiment of this invention relates to a single device incorporating tissue removal and clip placement capabilities, thus permitting single needle penetration for each tissue abnormality. Depending on the medical procedure application and patient circumstances, an integrated apparatus potentially enhances one or more of patient experience, physician satisfaction, procedural accuracy, procedural duration and safety. In other words, in one embodiment there is a safe, timely, efficient, effective and minimally invasive non-surgical breast core needle biopsy solution for the diagnosis of breast abnormalities by utilizing a single, integrated biopsy (tissue removal) and marker placement device for various medical procedures. Depending on application and patient circumstances, a multi-functional device might preferably streamline workflow, enhance procedural accuracy, reduce patient discomfort, ensure precise localization of biopsy sites for future imaging or surgical intervention, and reduce medical and environment waste.

Multiple embodiments and refinements of various aspects are disclosed and claimed herein. Some refinements are generally applicable to most, if not all, of these embodiments.

In one embodiment there is a single needle insertion breast biopsy and marker deployment apparatus. A needle extends longitudinally from a proximal end to a distal end having a sharpened tip. The needle defines a single internal passage connecting a proximal portal substantially adjacent to the proximal end and extending to a distal portal positioned substantially adjacent to the distal end. A casing encloses at least a portion of the proximal end of the needle. An actuator accessible via an outer portion of the casing is configured to be manipulated by an operator. The actuator has a first ready mode in which the distal portal is in fluid connection with a biopsy specimen chamber that is in fluid connection with the proximal portal. Activation of the actuator shifts from a first mode to a second mode. In the second mode the distal portal is in fluid connection with a marker conduit such that a marker positioned within the marker conduit may be deployed into the internal passage toward the distal portal.

In one refinement the marker conduit is at least partially enclosed within the casing, and the distal portal is substantially trough shaped.

In another refinement the actuator initiates a lateral translation of the marker conduit so as to at least partially align the marker conduit with the proximal portal.

In another refinement the actuator initiates an arc shaped translation of the marker conduit.

In another refinement the apparatus further comprises a flexible obturator configured to deploy the marker after a tissue sample is excised through the internal passage of the needle to the biopsy specimen chamber.

In another refinement a portion of the needle near the distal portal is a targeting band that is imaging visible to at least one imaging modality.

In another refinement the actuator opens a marker conduit portal that provides a fluid connection between the marker conduit and the internal passage of the needle toward the distal portal.

In another refinement the marker conduit and the internal passage of the needle are configured to be a substantially Y-shaped joint.

In another embodiment there is a single needle insertion breast biopsy and marking clip deployment interventional device. The device includes a biopsy needle having an internal passage extending longitudinally between a proximal end toward a sharpened tip distal end. There is a casing having an outer surface with a biopsy button and a separate marking clip button. The casing has a biopsy conduit and a marking clip conduit with each conduit being at least partially positioned within the casing along with the proximal end of the needle. A specimen container is fluidly connected by the biopsy conduit to the internal passage of the biopsy needle. A marking clip is positioned within the marking conduit. The biopsy conduit extends substantially longitudinally from and fluidly connects the specimen container to the internal passage of the biopsy needle. The marking clip is not capable of being deployed through the internal passage of the biopsy needle in a first mode. The marking conduit is fluidly connected to the internal passage of the biopsy needle such that the marking clip is capable of being deployed through the internal passage of the biopsy needle in a second mode.

In one refinement the biopsy needle includes a substantially trough shaped opening near the sharpened tip distal end.

In another refinement a Y-shaped joint fluidly connects the marking conduit to the biopsy conduit in the second mode.

In another refinement a portion of the device near the trough shaped opening is a targeting band that is imaging visible to at least one imaging modality.

In another refinement the device shifts from the first mode to the second mode by depressing the marking clip button.

In another refinement depressing the marking clip button causes lateral translation of the marking conduit in order to be fluidly connected and substantially longitudinally aligned with the internal passage of the biopsy needle.

In another refinement the lateral translation is arc-shaped.

In another embodiment there is a single needle insertion tissue excision and marking clip deployment at a tissue abnormality method. The method uses an at least partially common access channel. The skin is pierced and a distal penetrating biopsy tip of a needle is inserted to be substantially adjacent to a location of a tissue abnormality. A biopsy button is triggered. The tissue is excised from the abnormality and transferred to a specimen container through an internal passage of the needle into a biopsy conduit at least partially positioned within an outer casing. A clip deployment button is triggered to fluidly connect a marking clip conduit to the internal passage of the needle. This also deploys a marking clip within the marking clip conduit through the internal passage of the needle to a location substantially adjacent to the excised tissue abnormality. The biopsy and clip placement occur with a single needle insertion.

In one refinement triggering the clip deployment button accesses an opening defined by the biopsy conduit to permit marking clip deployment.

In another refinement triggering the clip deployment button laterally translates the marking clip conduit to be at least partially longitudinally aligned with the internal passage of the needle.

In another refinement the lateral translation is arc-shaped.

In another refinement triggering the biopsy button activates a cutting based excision through a trough opening substantially adjacent to the biopsy tip, and the excised tissue transfer to the specimen container is vacuum assisted.

### BRIEF DESCRIPTION OF THE FIGURES

FIG. 1 is a flowchart illustrating general aspects of a method of biopsy and clip marker deployment with a single needle penetration per tissue abnormality.

FIG. 2A illustrates a side cross-sectional view of an embodiment of a device in the ready state for use.

FIG. 2B illustrates a side cross-sectional view of an embodiment of a device in the pre-biopsy state.

FIG. 2C illustrates a side cross-sectional view of an embodiment of a device in the post-biopsy state.

FIG. 2D illustrates a side cross-sectional view of an embodiment of a device able to be operated remotely in the ready state for use.

FIG. 3A illustrates a side cross-sectional view of an embodiment of a device in the clip deployment ready state.

FIG. 3B illustrates a side cross-sectional view of an embodiment of a device in the post-clip (ready for removal) state.

FIG. 4 illustrates a side cross-sectional view of another embodiment of a device having side by side channels for biopsy and marker clip deployment.

### DETAILED DESCRIPTION OF THE PREFERRED EMBODIMENTS

For purposes of promoting an understanding of the principles of the invention, reference will now be made to the embodiments illustrated in the drawings and specific language will be used to describe the same. It will nevertheless be understood that no limitation of the scope of the invention is thereby intended, such alterations and further modifications in the illustrated device, and such further applications of the principles of the invention as illustrated therein being contemplated as would normally occur to one skilled in the art to which the invention relates.

Using two separate breast interventional devices (one for biopsy and another for clip placement) was recognized as correlating with a number of potential limitations and/or disadvantages. As a first example, switching between the two separate devices increases procedure time with potential increased patient discomfort and anxiety. The resulting increased procedure duration can be associated with increased bleeding. As another example, switching between the two separate devices increases the risk of accidental needle stick for the user of the devices (sharp tips are present on both the biopsy device and marker clip placement device). Additionally, biopsy procedure preparation time is increased by the need to set up for two separate devices. As most users of each of the two separate devices have a favored hand in which to utilize the device, the user must also receive, use, return and receive, use and return each device in their favored hand, adding further time and procedural complexity. Furthermore, administratively, inventory tracking and operative certification of multiple devices are also required.

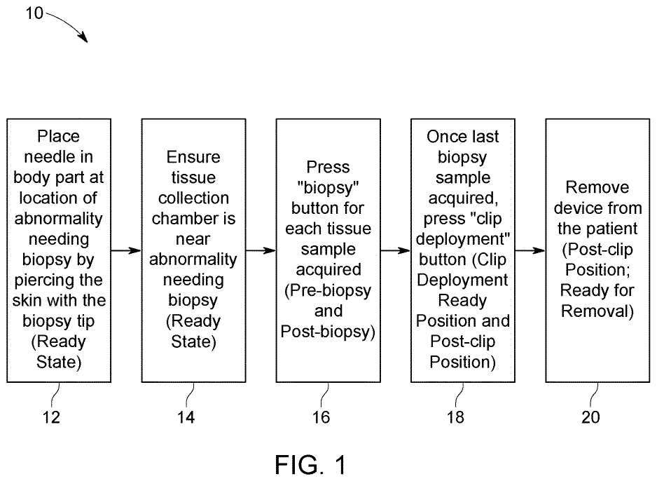

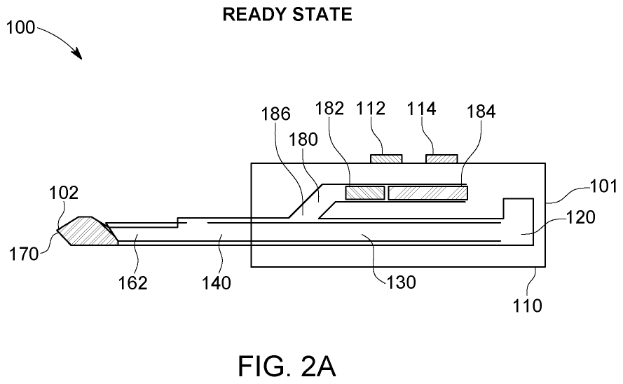

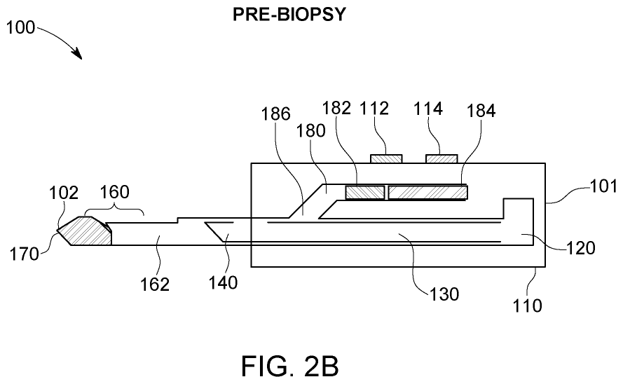

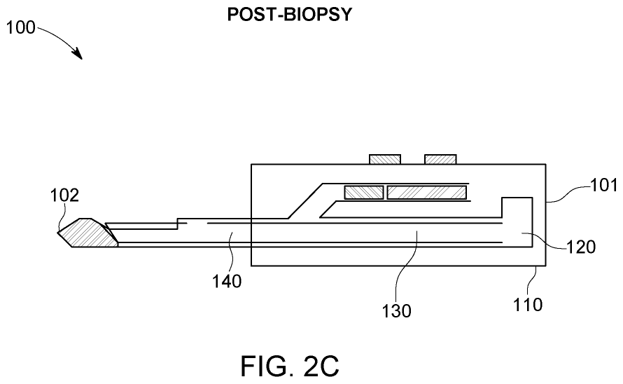

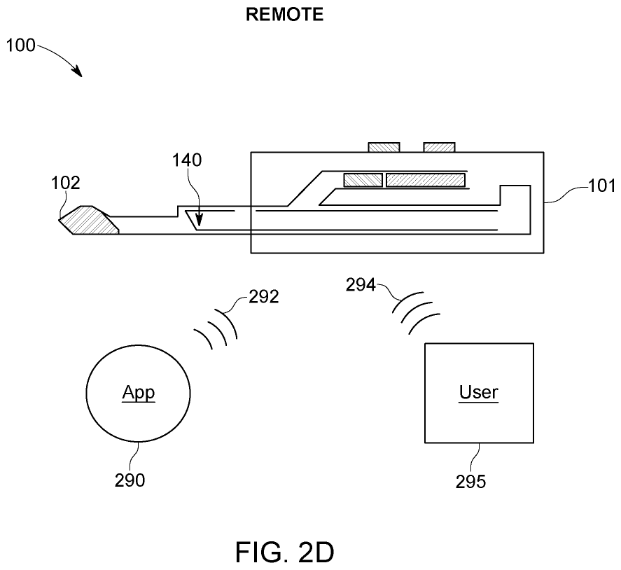

With reference to FIG. 1, there is a flow chart 10 illustrating general aspects of the operation and use of various embodiments of one or more devices disclosed herein. The step 12 involves the device in the ready state (see FIG. 2A for one example). Step 12 includes placing a distal penetrating tip of a needle in a portion of patient anatomy at a location of the abnormality by piercing the skin with the biopsy tip. The next step 14 also involves the device in the ready state. During step 14 the user preferably ensures the tissue collection chamber is sufficiently proximate to the abnormality to be biopsied to permit sample excision at or near the biopsy tip and transfer to the collection chamber.

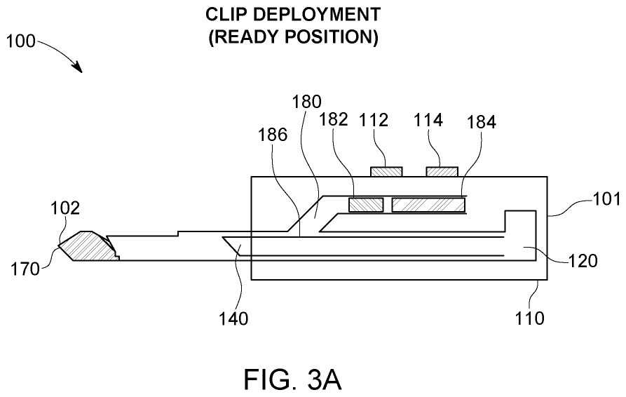

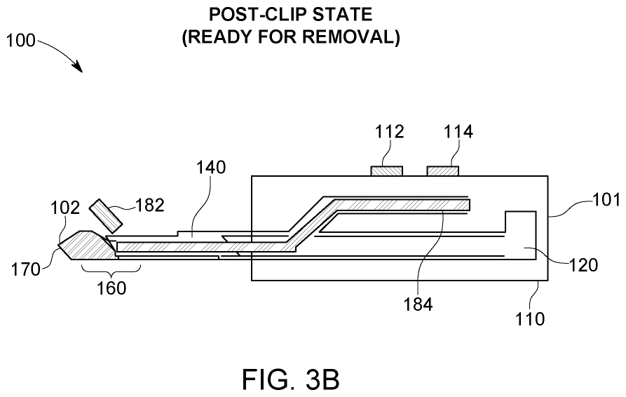

The next step 16 involves the device in the pre-biopsy state (see FIG. 2B for one example) and/or post-biopsy state (see FIG. 2C for an example). Step 16 entails pressing the biopsy button for each tissue sample to be acquired. The subsequent step 18 involves the device in the clip deployment ready position (see FIG. 3A) and post-clip position (see FIG. 3B). After the last biopsy sample of a procedure for one identified tissue abnormality is acquired, step 18 includes pressing the marker clip deployment button. Finally, in general terms, there is step 20 that includes removing the multi-function (breast) interventional device from the patient (see FIG. 3B for post-clip position).

With reference to FIGS. 2A-2D there are shown various features of one embodiment of a breast biopsy interventional device 100 extending longitudinally from a proximal end 101 toward a distal end 102. The distal end 102 includes a cutting or biopsy tip 170 that is sufficiently sharp to penetrate tissue. In FIG. 2A the device is in the ready state. In FIG. 2B the device is in the pre-biopsy mode, and in FIG. 2C the device is in the post-biopsy mode. In FIG. 2D the device is operated remotely via app, software or a remote user. Apparatus 100 is intended in one application of the present invention to be longitudinally inserted into breast tissue as guided by an appropriate imaging modality. While not described in detail for use in conjunction with the same, the present invention is contemplated for use in vacuum-assisted biopsy applications.

The apparatus 100 possesses an outer casing 110. Externally accessible to the user on the outer casing 110 is a biopsy button 112 and a clip deployment button 114. The term “button” or “actuator” is used herein in a broad sense. “Button” or “actuator” is intended to encompass a variety of activation mechanisms including, but not limited to, a latch, switch, trigger, rotating knob, roller ball, or key, or other means for activating a desired functionality during the procedure by a user, as well as the more common understanding of a substantially circular shaped protrusion or protuberance that is depressed, contacted, triggered, or slid (or perhaps even pulled upon) to activate the engagement and release. Additionally, in one or more embodiments, where the equivalent of the button or actuator is digital in operation and application, software is utilized to operate a digital switch to activate, engage and release the clip as described herein. Further, in one or more additional embodiments, a digital switch of the device is able to be operated remotely by a user, controlling operating system, or via an artificial intelligence system.

At least partially within outer casing 110 is a specimen container 120. The outer casing 110 is preferably configured to be held by a user's hand, and casing 110 might be either reusable or disposable. Also positioned at least partially within the outer casing 110 is a first channel or biopsy channel 130, and a second channel or marker channel 180. Each channel might be defined by the interior of the outer casing 110. Alternatively, the outer casing 110 might be at least partially hollow and the channels defined by separate wall passages extending between the proximal end toward the distal end. For example, as illustrated in FIGS. 2A-2C the inner casing 140 with cutting tip defines the biopsy channel and extends through the distal portion of the outer casing 110 to tissue collection chamber 160 that includes a trough section 162. The inner casing then further extends via sharpened biopsy tip 170 and at distal end 102.

The outer casing 110 thus substantially encompasses the specimen container 120 and at least a portion of the inner casing 140. When tissue samples are removed from the patient, they are collected in specimen container 120, which is removable after the biopsy procedure. Specimen container 120 is fluidly connected to the biopsy channel 130 positioned within inner casing 140, and is preferably a polymer that does not distort or interfere with the imaging modality being used. It is understood, however, that portions of the breast biopsy interventional device 100 might be imaging visible (visibility on any imaging modality, e.g. x-ray, US, and MRI). In particular, imaging visible portions near the distal end, such as that being adjacent or on the cutting tip 170 (as with a “targeting” band), and also a portion or the entirety of each marker clip 182. While not presently designated in the figures, it will be understood by those of ordinary skill in the art that portions of interventional device 100 might be imaging visible, and thus act as a targeting band in inserting the distal end 102 to be positioned adjacent to the tissue to be sampled.

Inner casing 140 forms the wall of the conduit (biopsy channel 130) through which collected tissue samples travel from the tissue collection chamber 160 to the specimen container 120. The distal end 102 features a cutting-opened biopsy tip 170. In one embodiment the inner casing 140 includes an opening to form a substantially Y-shaped joint 186 that fluidly connects the marker channel 180 to biopsy channel 130. That opening permits flexible obturator 184 to impel marker clip 182 (usually comprising metal or having a metallic component) to the volume defined by the excised tissue after tissue removal is completed.

During the biopsy, the tissue collection chamber 160 section of device 100 is open along at least a portion of its “top” (an arbitrarily assigned direction that is toward the tissue to biopsy) aspect, pulling the tissue into it, and the cutting tip of the inner casing 140 will shave off a piece of tissue. After cutting, the tissue travels through the inner casing into the specimen container 120 via biopsy channel 130. Depressing biopsy button 112 might preferably cause the inner casing 140 with the cutting tip to automatically retract before automatically advancing to cut the tissue.

The outer casing 110 further contains at least partially therein marker channel 180. Marker channel 180 contents include marker clip 182 and flexible obturator 184. In one illustrated embodiment the marker channel 180 is fluidly connected to the main inner casing 140 utilizing a Y-type junction 186. The marker channel is preferably closed during biopsy; however, it communicates with the biopsy channel 130 of inner casing 140 when the hole of the inner casing 140 is aligned with the hole of the marker channel 180. The marker clip 182 is deployed from the marker channel 180 by pressing clip deployment button 114.

Flexible obturator 184 in marker channel 180 deploys (preferably a semi-automatic mode of operation) the marker clip 182 by pushing it from the marker channel 180 through the Y-shaped connection 186. The flexible obturator 184 then further deploys the marker clip 182 through the opening in the inner casing 140, into the tissue collection chamber 160, and into the patient when clip deployment button 114 is pressed. The flexible obturator 184 does not exit the tissue collection chamber 160, and instead its distal end typically remains inside the tissue collection chamber 160. In one refinement the flexible obturator at least partially comprises metal, and might be a coil spring.

After clip deployment button 114 is pressed, the biopsy button 112 preferably ceases to function. In other words, after the biopsy (tissue removal) is completed, the clip would be deployed. As an example, in one embodiment pressing clip deployment button 114 might transmit an electronic signal that would be sent to biopsy button 112. Additionally, in some refinements the change in configuration of the device in the clip placement position precludes activating the mechanical function of the biopsy capability. For example, the inner mechanical components of the biopsy portion might not align with the biopsy button.

With reference to FIG. 2D, the apparatus of the present invention 100 is able to connect remotely with an application 290 which is operated by a user 295. The connectivity 292 as between the apparatus 100 and the app 290 may be via one of any of a plurality of connective means such as Bluetooth, internet, 5G, wireless mobile systems, or a connected wire or cable system. Alternatively, the app 290 may also be a logic control device which receives commands from a command source providing instruction to the apparatus as to which state and activation step to undertake.

With reference to FIG. 3A, the apparatus of the present invention 100 is in a clip deployment state where it is positioned for the clip 182 to be inserted. In one embodiment, a user may deploy the marker clip 182 from the marker channel 180 by pressing clip deployment button 114. Operatively, the flexible obturator 184 in marker channel 180 is then activated to the marker clip 182 by pushing it from the marker channel 180 through the Y-shaped connection 186.

With reference to FIG. 3B, the apparatus of the present invention 100 is in a post-clip state (i.e., the clip 182 has been deployed from the device). The flexible obturator 184 was activated to deploy the marker clip 182 through the opening in the inner casing 140, through the tissue collection chamber 160, and into the patient when clip deployment button 114 is pressed. The flexible obturator 184 does not exit the tissue collection chamber 160, and instead its distal end typically remains inside the tissue collection chamber 160. In one refinement the flexible obturator at least partially comprises metal, and might be a coil spring.

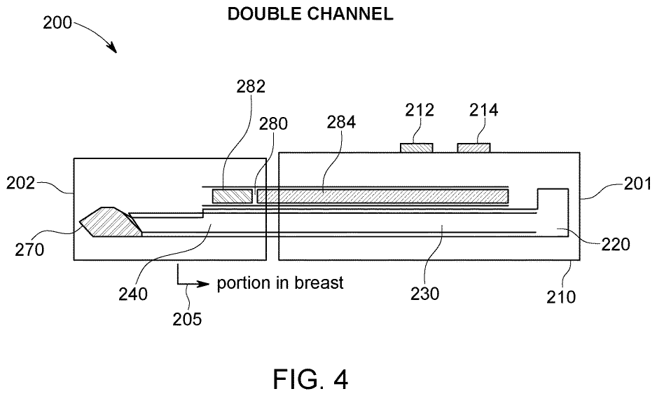

With reference to FIG. 4 there is illustrated an alternative embodiment of interventional device 200 having a distal end 202 and a proximal end 201. In at least some respects, while adequate for a fully-unitary operation, this embodiment may be considered a less desirable alternative. It includes two wholly separate channels (one for biopsy 230 and one for clip placement 280) instead of the prior at least partially “common” channel embodiment. This increases the device diameter in the patient tissue, and potentially increases patient discomfort and/or bleeding. A larger portion 205 of the device 200 in this embodiment would be positionally placed into the breast area for procedural success. This might also limit accessibility to small breast abnormalities in some parts of the breast, such as near the chest wall. Having now briefly overviewed general aspects of a less preferred embodiment in some respects, further details will be discussed with reference to FIG. 4.

Breast biopsy interventional device 200 extends longitudinally from a proximal end 201 toward a distal end 202. The distal end 202 includes a cutting or biopsy tip 270 that is sufficiently sharp to penetrate tissue. The apparatus 200 is intended to be longitudinally inserted into a breast under the guidance of an appropriate imaging modality. While not described in detail for use in conjunction with the same, the present embodiment is also contemplated for use with vacuum-assisted biopsy applications.

The apparatus 200 includes an outer casing 210. Externally accessible to the user on the outer casing 210 is a biopsy button 212 and a clip deployment button 214. At least partially within outer casing 210 is a specimen container 220. The outer casing 210 is preferably configured to be held by a user's hand, and casing 210 might be either reusable or disposable. Also positioned at least partially within the outer casing 210 is a first channel or biopsy channel 230, and a substantially side by side second channel or marker channel 280. Each channel might be defined by the interior or the outer casing 210. Alternatively, the outer casing 210 might be at least partially hollow and the channels defined by separate passages extending between the proximal end toward the distal end. The channels are not fluidly connected, but instead substantially parallel in the longitudinal direction.

Conduit 240 is the portion through which collected tissue samples travel from the tissue collection chamber to the specimen container 220. The distal end features a cutting tip 270. Flexible obturator 284 is present to impel marker clip 282 (usually comprised of metal or having a metallic component) to the volume defined by the excised tissue after tissue removal is completed.

Further alternative embodiments are contemplated as within the scope of the invention beyond the previously described Y-joint, or the less preferred parallel channels extending to the distal end. A related example is that instead of being adjacent conduits both within a substantially cylindrical passage of the core biopsy needle, the biopsy channel might be an inner substantially cylindrical passage and the marker passage might be an annulus defined between the external surface of the biopsy channel and the internal surface of the core biopsy needle.

As another example, the separate biopsy channel and marker clip channel might be substantially parallel but each separately and alternatively fluidly connected when aligned with the core biopsy needle inserted into tissue. In other words, the separate channels are at different times aligned longitudinally with the passage through the needle. In one refinement these two channels are shifted laterally in a direction defined by horizontal and vertical axes substantially perpendicular to the longitudinal direction. In a further refinement these channels may be aligned with the longitudinal passage through the needle by being rotated on a cylindrical mechanism in a manner similar to a standard revolver rotating to align a cartridge with the device's barrel. Alignment of the channels in each instance may be further improved by inclusion of a beveled interface. Movement from a biopsy mode to a marker clip mode (with consequent lateral and/or rotational movement) might be activated by the marker clip deployment button or possibly via a separate trigger.

The use of tissue markers after percutaneous breast biopsy has become routine. Studies have found that in over 75% of stereotactic and ultrasound biopsies, complete imaging removal of an abnormality is achieved (Fine RE, Israel PZ, Walker LC, Corgan K R, Greenwald L V, Berenson J E, Boyd B A, Oliver M K, McClure T, Elberfeld J. A prospective study of the removal rate of imaged breast lesions by an 11-gauge vacuum-assisted biopsy probe system. Am J Surg. 2001 October; 182(4): 335-40. Salazar J P, Miranda I, de Torres J, Rus M N, Espinosa-Bravo M, Esgueva A, Salvador R, Rubio I T. Percutaneous ultrasound-guided vacuum-assisted excision of benign breast lesions: A learning curve to assess outcomes. Br J Radiol. 2019 February; 92(1094): 20180626). In addition, with benign lesions, tissue markers can remain in the breast indefinitely, facilitating identification and follow-up.

Thus, as previously discussed, a localizing marking clip or gel marker is placed to mark as required for surgical excision. The marker can be visualized on mammography, ultrasound, and/or MRI. The marking clip allows identification of the biopsy site on subsequent mammograms. The marker clip is usually metallic, measures 2-3 mm, and typically comprises either titanium or stainless steel, though nonmetallic carbon and ceramic markers exist. Some marker clips are embedded in a bioabsorbable substance, and/or possess polymeric embedding agents to improve visibility under various imaging techniques, possibly increase hemostasis by direct pressure on the biopsy cavity, and decrease risk of clip migration.

In another refinement the site markers may also possess different characteristics to distinguish between them, of potentially significant value when multiple marker clips are deployed. As one example, the marking clips might possess differential characteristics as to shape and/or cross-section. For example, it may be shaped similar to a staple and possess two prong elements joined by a connecting member. A second marking clip may also vary in configuration to possess one or more different cross-sectional shapes in the imaging modality utilized. This variation would permit recordation of the marking clips associated with different biopsy samples based on something other than their position in the human anatomy as recorded by the imaging modality. Such position may be altered to the extent the marking clip might migrate or be displaced after initial deployment. Alternatively, the marking clip may also include some form of RFID or Bluetooth-emitting technology for post-procedure identification as associated with a particular biopsy sample where it may also contain data associated with the patient, procedure and medical diagnosis.

It will be understood by those of skill in the art that embodiments of the present invention will preferably not have internal reusable parts. Indeed, it is important not to reuse even a common channel on the same patient for a different internal volume in the breast. The possibility of pathology tissue contamination occurring far outweighs any potential benefit in cost-savings. For example, if two abnormalities are present in the same breast, two different disposable proposed devices should be used. Nonetheless, operatively, the present invention is capable of reuse and repetitive operation.

The invention disclosed herein is intended for use with a variety of imaging modalities. Such imaging modalities include, but are not limited to, ultrasound-guided and stereotactic-guided breast biopsies. While the devices and method discussed herein primarily refer to breast biopsy, one or more core precepts of the disclosed invention are not limited to that portion of the human anatomy. Nor do the claims preclude application to other anatomy features unless explicitly limited to the same.

Small structural differences in design might nonetheless present significant improvement compared to the state of the art. The advantageous ramifications to the disclosed embodiments are thus now discussed herein.

The procedure improves upon traditional breast biopsies. Pre-procedure set-up time will be of decreased duration. Intra-procedure duration is faster and safer. Less post-procedure environmental waste might be generated from product packaging (single product package versus two product packages). Patient experience will be improved since the procedure will be shorter and more comfortable with a reduction in post-procedure complications. Patient satisfaction (“patient at the center”) is a key component of healthcare, and the invention improves patient comfort.

Less waste might be an additional motivation from an environmental perspective. Existing deployment devices for clip placement are single use disposable devices. The tissue marker clip is typically only 2-4 mm in maximum diameter. The clip placement device is not reusable and a significant amount of medical waste occurs (device itself and packaging material) with each procedure.

The multi-function device provides more accurate clip placement as the cutting tip and passage thereto remain in situ for the entirety of the procedure. Additionally, the multi-function device should reduce bleeding complications and accidental user needle sticks.

While the invention has been illustrated and described in detail in the drawings and foregoing description, the same is to be considered as illustrative and not restrictive in character, it being understood that only the preferred embodiments have been shown and described and that all changes and modifications that come within the spirit of the inventions are desired to be protected. It should be understood that while the use of words such as preferable, preferably, preferred or more preferred utilized in the description above indicate that the feature so described may be more desirable, it nonetheless may not be necessary and embodiments lacking the same may be contemplated as within the scope of the invention, the scope being defined by the claims that follow. In reading the claims, it is intended that when words such as “a,” “an,” “at least one,” or “at least one portion” are used there is no intention to limit the claim to only one item unless specifically stated to the contrary in the claim. When the language “at least a portion” and/or “a portion” is used the item can include a portion and/or the entire item unless specifically stated to the contrary.

What is claimed is:

1. A single needle insertion breast biopsy and marker deployment apparatus comprising:
a needle extending longitudinally from a proximal end to a distal end having a sharpened tip, the needle defining a single internal passage connecting a proximal portal at the proximal end and extending to a distal portal positioned substantially adjacent to the distal end;
a casing enclosing at least a portion of the proximal end of the needle;
an actuator accessible via an outer portion of the casing and configured to be manipulated by an operator of the single needle insertion breast biopsy and marker deployment apparatus; and
wherein the actuator has a first ready mode in which the distal portal is in fluid connection with a biopsy specimen chamber that is in fluid connection with the proximal portal, and wherein activation of the actuator shifts from a first mode to a second mode in which the distal portal is in fluid connection with a marker conduit such that a marker positioned within the marker conduit may be deployed into the single internal passage toward the distal portal.

2. The apparatus of claim 1, wherein the marker conduit is at least partially enclosed within the casing, and wherein at least a portion of the distal portal is substantially trough shaped.

3. The apparatus of claim 1, wherein the actuator initiates a lateral translation of the marker conduit that at least partially aligns the marker conduit with the proximal portal.

4. The apparatus of claim 3, wherein the actuator initiates an arc shaped translation of the marker conduit.

5. The apparatus of claim 3, further comprising a flexible obturator configured to deploy the marker after a tissue sample is excised through the internal passage of the needle to the biopsy specimen chamber, and wherein a portion of the needle near the distal portal is visible on at least one imaging modality.

6. The apparatus of claim 1, wherein the actuator opens a marker conduit portal that provides a fluid connection between the marker conduit and the internal passage of the needle.

7. The apparatus of claim 6, wherein the marker conduit and the internal passage of the needle are configured to be a substantially Y-shaped joint.

8. The apparatus of claim 7, further comprising a flexible obturator positioned within the marker conduit for advancing the marker toward the distal portal, and wherein a portion of the needle near the distal portal is visible on at least one imaging modality.

9. A single needle insertion breast biopsy and marking clip deployment interventional device comprising:
a biopsy needle having an internal passage extending longitudinally between a proximal end toward a sharpened tip distal end;
a casing having an outer surface with a biopsy button and a separate marking clip button, wherein the casing has a biopsy conduit and a marking clip conduit with each conduit and the proximal end of the needle being at least partially positioned within the casing;
a specimen container fluidly connected by the biopsy conduit to the internal passage of the biopsy needle;
a marking clip positioned within the marking conduit;
wherein the biopsy conduit extends substantially longitudinally from and fluidly connects the specimen container to the internal passage of the biopsy needle and the marking clip is not capable of being deployed through the internal passage of the biopsy needle in a first mode, and wherein the marking conduit is fluidly connected to the internal passage of the biopsy needle such that the marking clip is capable of being deployed through the internal passage of the biopsy needle in a second mode.

10. The device of claim 9, wherein the biopsy needle includes a substantially trough shaped opening near the sharpened tip distal end.

11. The device of claim 10, wherein a Y-shaped joint fluidly connects the marking conduit to the biopsy conduit in the second mode.

12. The device of claim 10, wherein a portion of the device near the trough shaped opening is imaging visible to at least one imaging modality.

13. The device of claim 12, wherein the device shifts from the first mode to the second mode by depressing the marking clip button.

14. The device of claim 13, wherein depressing the marking clip button causes lateral translation of the marking conduit such that the marking conduit is fluidly connected and substantially longitudinally aligned with the internal passage of the biopsy needle.

15. The device of claim 14, wherein the lateral translation is arc-shaped.

16. A method of single needle insertion tissue excision and marking clip deployment at a tissue abnormality using an at least partially common access channel comprising:
piercing the skin and inserting a distal penetrating biopsy tip of a needle to be substantially adjacent to the tissue abnormality;
triggering a biopsy button and excising tissue from the abnormality and transferring the excised tissue from a distal portal of the needle and through an internal passage of the needle to a proximal portal of the needle and onward to a specimen container via a biopsy conduit at least partially positioned within an outer casing;
triggering a clip deployment button and fluidly connecting a marking clip conduit to the internal passage of the needle and deploying a marking clip within the marking clip conduit at least partially through the internal passage of the needle to a location substantially adjacent to the excised tissue; and,
wherein biopsy and marking clip placement occur with a single needle insertion.

17. The method of claim 16, wherein triggering the clip deployment button accesses an opening defined by the biopsy conduit to permit marking clip deployment through the opening into the internal passage of the needle.

18. The method of claim 16, wherein triggering the clip deployment button laterally translates the marking clip conduit to be at least partially longitudinally aligned with the internal passage of the needle.

19. The method of claim 18, wherein the lateral translation is arc-shaped.

20. The method of claim 16, wherein triggering the biopsy button activates a cutting based excision through a trough-shaped distal portal substantially adjacent to the biopsy tip, and wherein the excised tissue transfer to the specimen container is vacuum assisted.
